(19) United States
(12) Patent Application Publication
(10) Pub. No.: US 2006/0015147 A1
(43) Pub. Date: Jan. 19, 2006

(54) Apparatus for controlling the generation of electric fields

(75) Inventors: Bertil R. R. Persson, Lund (SE); Bernt J. Bohmer, Stehag (SE); Bo H.G. Thorvinger, Malmo (SE)

Correspondence Address: LADAS & PARRY, 26 WEST 61ST STREET, NEW YORK, NY, 10023, US

(73) Assignee: ADITUS MEDICAL AB.

(21) Appl. No.: 11/204,258

(22) Filed: Aug. 15, 2005

Related U.S. Application Data
(63) Continuation of application No. 09/601,751, filed on Aug. 7, 2000.

(30) Foreign Application Priority Data
Mar. 31, 1998 (SE) 9801139-8

(51) Int. Cl. A61N 1/39; A61N 1/18

(52) U.S. Cl. 607/5; 607/72

(57) ABSTRACT

An apparatus (60) according to the present invention includes a voltage generator (1) for generating brief voltage pulses for the impression of voltage on electrodes (6, 15, 16, 24) included in the apparatus, and a measurement unit (50) which is coupled to the electrodes. These are designed to be secured at or inserted in tissue in a restricted region of a human or an animal in order therebetween to form electric fields in the tissue. The measurement unit (50) is disposed to determine the impedance between the electrodes which is substantially determined by the electric properties of the tissue which is located between the electrodes. A registration and calculator device (10) forms a control unit which, based on the impedance determined by the measurement unit, controls the output voltage of the voltage generator such that the electric field which is formed in the tissue always has a predetermined value. The treatment with the electric field realizes a perforation of cell membranes in the tissue which thereby permits the passage of substances fed to the body (e.g. cytostatic or genetic material).

This is a continuation of copending application Ser. No. 09/601,751 filed on Aug. 7, 2000 which is a 371 of PCT/SE99/00511 filed Mar. 30, 1999 claims the benefit thereof and incorporates the same by reference.

The present invention relates to an apparatus for generating pulses of electric fields in a restricted area of a human or an animal according to the preamble to the appended independent claim.

The therapy forms which are routinely employed in modern medical care for tumor therapy are examples of treatment types where the outcome of such treatment is unsatisfactory. For example, in tumor therapy unsuccessful attempts are often made to achieve local tumor control, which is the cause of mortality of approximately 30% of cancer patients. It is, therefore, important to develop a new and improved technique for local and regional tumor treatment.

In today's medical-care, surgery, chemotherapy and radiation therapy, also known as radiation treatment, or combinations hereof are the most commonly employed methods for treating malignant tumors. Approximately every second patient suffering from infiltrating cancer is treated with radiation therapy, but only roughly half of the patients are cured. This failure is, on the one hand, the cause of the presence of wide-spread disease (distal metastasis) or relapses (the return of tumors in the treated area), and on the other hand because certain types of tumor are resistant to radiation treatment or chemotherapy.

With varying success, attempts have been made to reinforce and improve the efficiency of radiation therapy in sterilizing tumors. For example, use has been made of more sophisticated radiation therapy techniques, such as stereotactic treatment, “conformal radiotherapy”, of altered fractioning or added pharmaceuticals to increase the radiation sensitivity of the tumors.

Use is also made of heat as an adjuvant ionizing radiation, which, for certain, tumor forms, may increase the number of complete remissions by up to a factor of two.

Also in certain purely medically treated diseases in local organs, the outcome of treatment is occasionally insufficient. It is obvious that, in addition to the wishes which exist regarding improved techniques for treating, for example, tumors, there are not only wishes but also needs for a more efficient technique for treating certain other diseases. In, for example, the local treatment of local organs or tumors, it is a major advantage if, on each treatment occasion, it is possible to adapt the intensity of the treatment to suit the status of the tissue in the local region or in the organ being treated.

According to the present invention, use is made of a series of brief high voltage pulses for generating electric fields in the local region or in the organ which is to be treated. In the continuation of this description, use will also be made of the expression High Voltage Impulse Therapy, occasionally abbreviated to HVIT.

The treatment with electric fields realizes a perforation of the cell membranes which thereby allow the passage of substances (e.g. cytostatic or genetic material) added to the body. The treatment involves increased inflow of therapeutic substances, whereby the effects of chemotherapy are amplified. The outflow of specific substances out of, for example, tumor cells moreover often realizes a stimulation of the immune system. In total dielectric collapse, the result is often achieved that the cells are sterilized directly by the electric fields formed by the high voltage pulses. In clinical experiments, the method has proved to be effective in combination with cytostatics (Bleomycin) for, for example, treating melanoma and tumors in the neck, head, liver, pancreas and lungs.

In HVIT, the treatment result is determined by the number and duration of the high voltage pulses to which the tissue is subjected and how high electric field forces the impressed pulses create in the tissue, as well as the form or frequency the pulses possess. In order to achieve an effective and dependable treatment, it must be possible to control all of these physical parameters. Biological properties which affect the treatment result are, among other things, the electric conductive capacity of the tissue, its dielectric properties, the cell sizes and the structures of the cell membranes. All of these properties vary between different tissues. In order to achieve optimum treatment effect, it is therefore necessary to measure how the electric properties of the tissue change between each high voltage pulse or between the series of pulses, i.e. to establish when the cells are sufficiently perforated.

In previously employed HVIT, it was not possible to monitor when the tissue was sufficiently perforated, i.e. when the treatment was completed, which entailed that the tissue was occasionally undertreated and occasionally overtreated. This involved a degree of uncertainty in the treatment result. A typical HVIT treatment according to prior art techniques entails that an applicator was placed over the tissue which was intended for treatment. The high voltage generator was, for example, set such that the outgoing voltage corresponded to a field force in the target volume of approx. 1300 V/cm. The treatment was completed with a fixed number of pulses which it was known normally gave the desired result. The weaknesses in this procedure were, on the one hand, that the size of the electric field which the generator in reality generated in the tissue of the target volume was unknown, and, on the other hand, that it was not possible to assess when the treatment was sufficient.

The present invention relates to an apparatus which includes mechanical devices for subjecting a tissue within a restricted region or an organ in a person or an animal for one or more pulses of an electric field at a field strength, configuration, duration and frequency adjustable for the relevant treatment occasion. The expression “duration” relates to both the length of the pulses and the number of pulses, the expression “frequency” relates to both how often the pulses are repeated and the frequency with which the field alternates during an ongoing pulse.

The characterizing clause of the appended independent claim discloses a technique which entails a substantial improvement to the efficiency of surgery, chemotherapy and radiation therapy. The technique is also applicable within modern molecular medicine where substances and genetic DNA sequences which are to be introduced into tissue cells are customized.

Further expedient embodiments of the present invention are disclosed in the appended subclaims.

The present invention will now be described in greater detail hereinbelow, with reference to a number of Figures, in which:

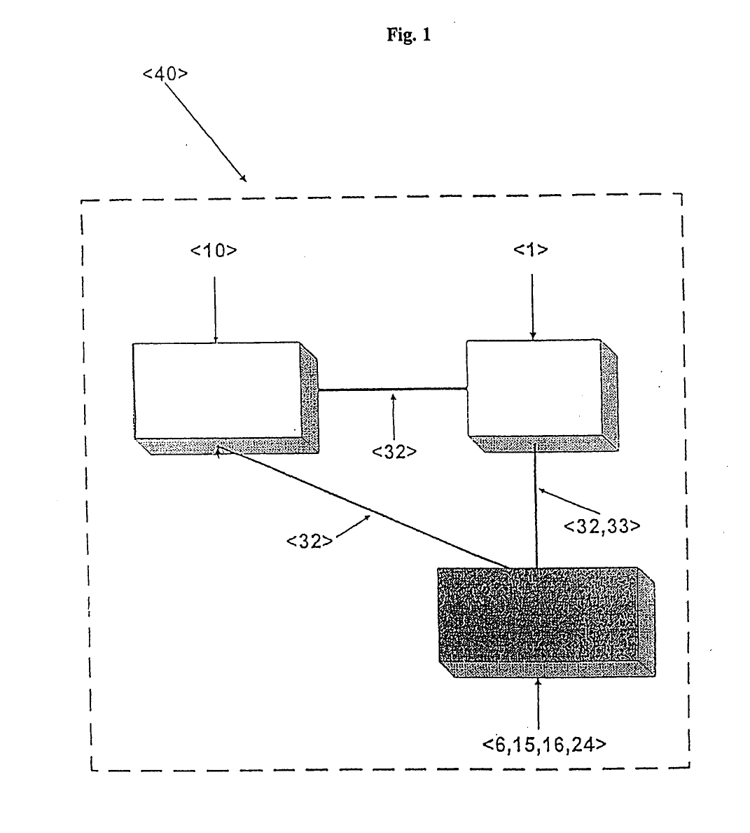

FIG. 1 is a block diagram of a fundamental apparatus for applying electric fields in a restricted region of a person or an animal;

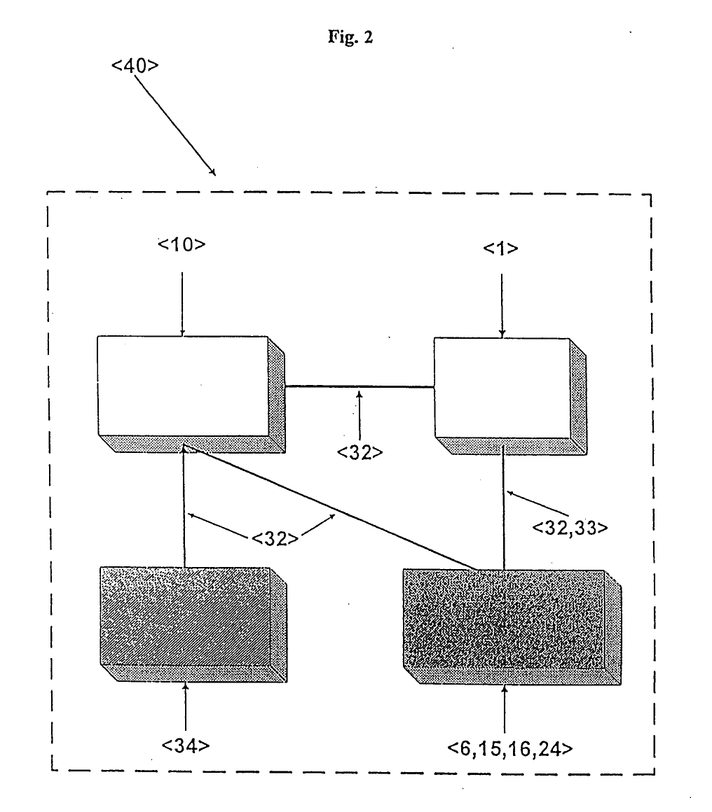

FIG. 2 is a block diagram of a fundamental apparatus for applying electric fields and/or ionizing radiation in a restricted region of a human or an animal;

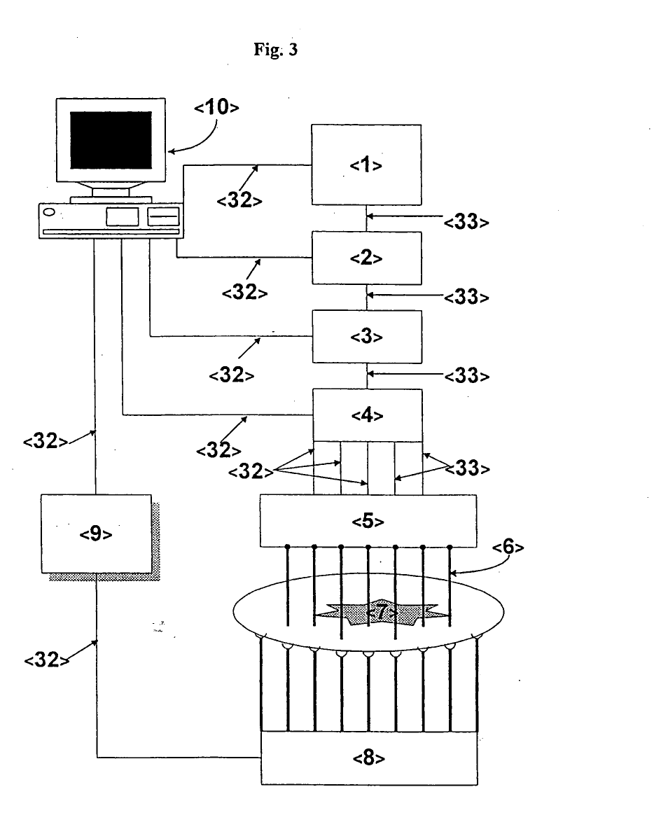

FIG. 3 is a block diagram of one embodiment of a combination of devices for forming electric fields in a restricted region of a human or an animal;

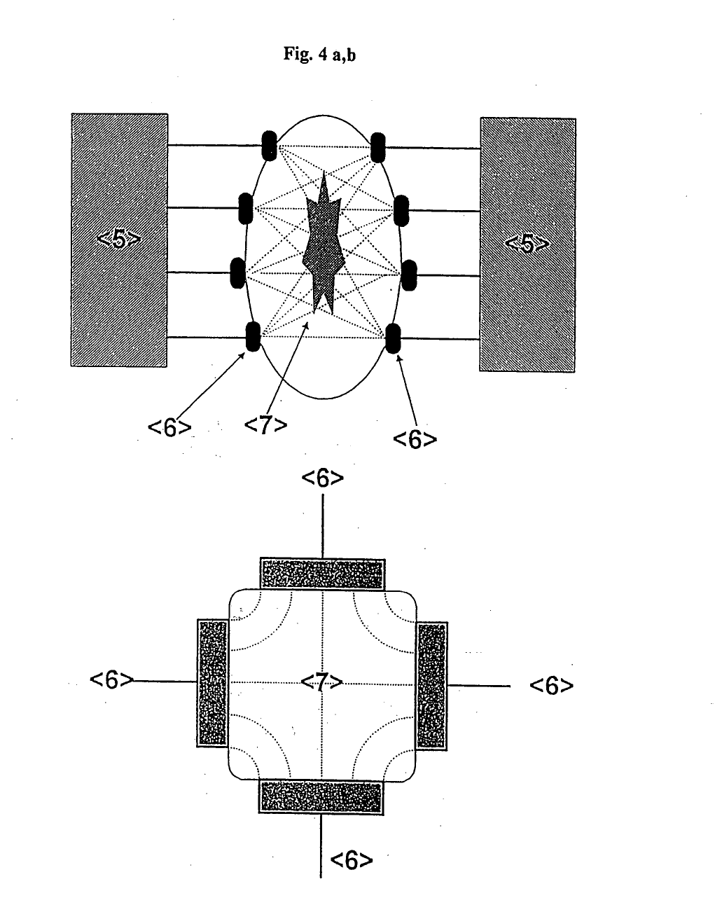

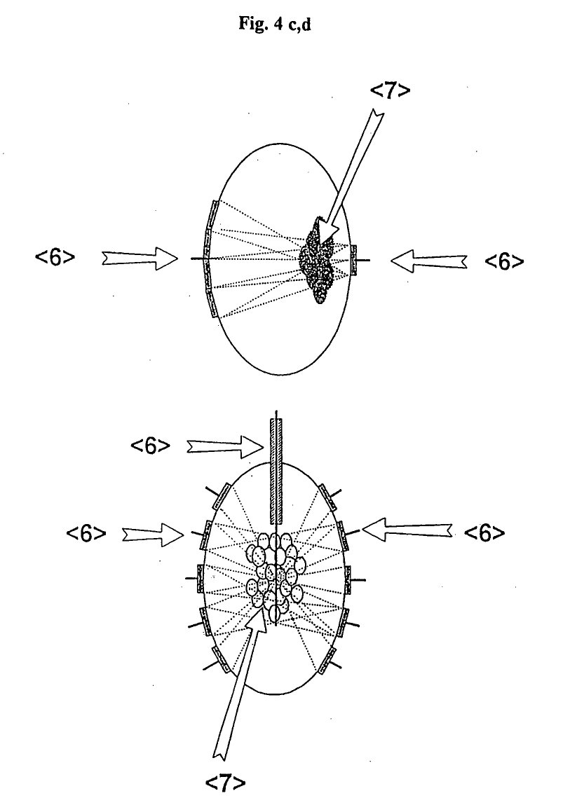

FIGS. 4a-d are embodiments of electrode applicators for external treatment of tissue;

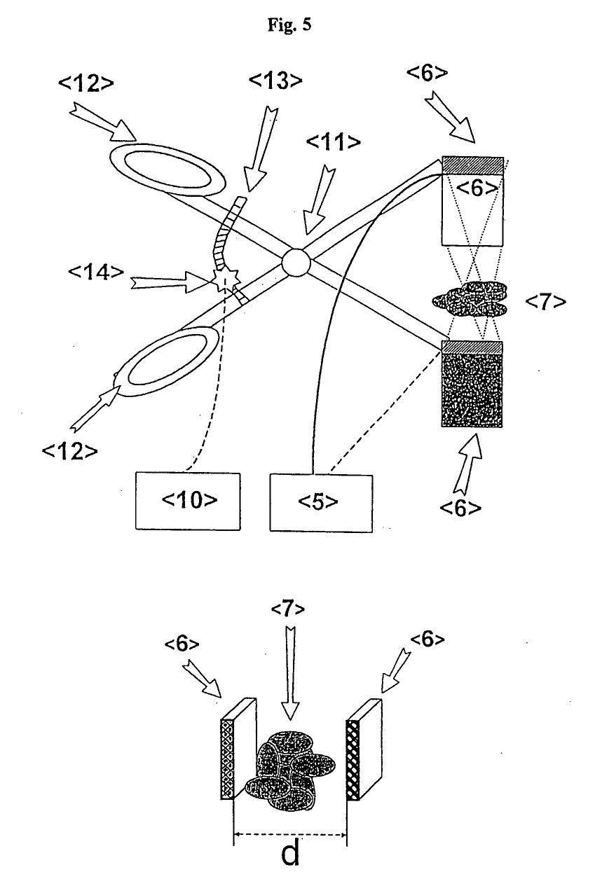

FIG. 5 shows one embodiment of an electrode applicator for intraoperative treatment of, for example, tumors and superficial tumor nodules;

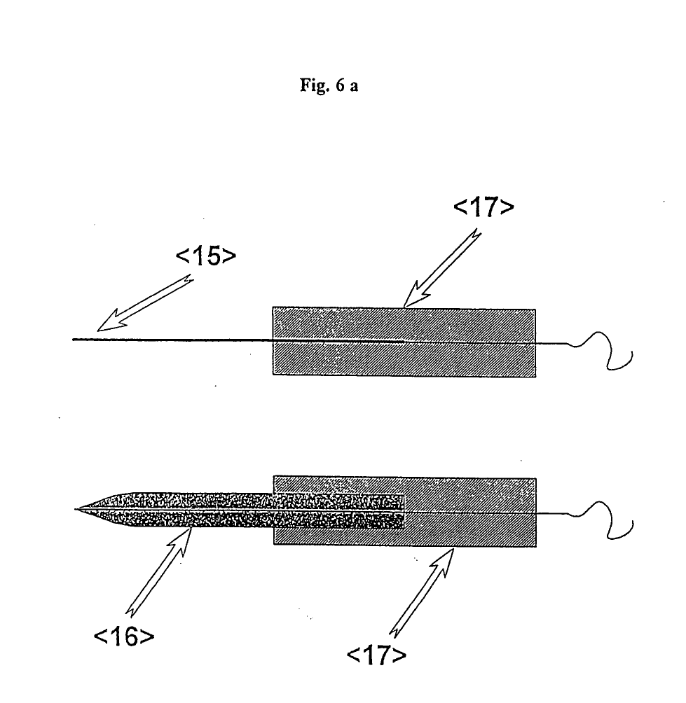

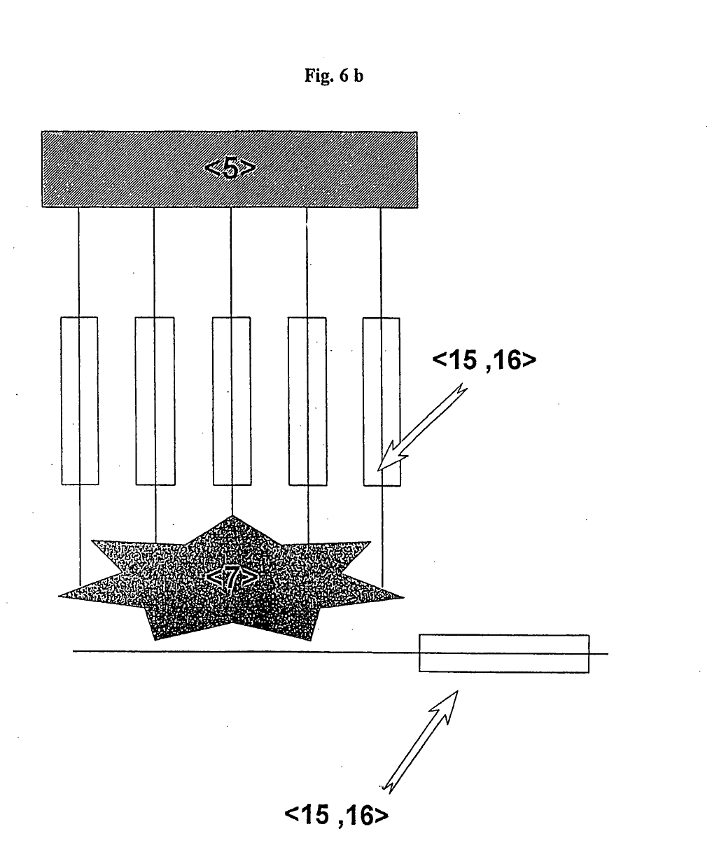

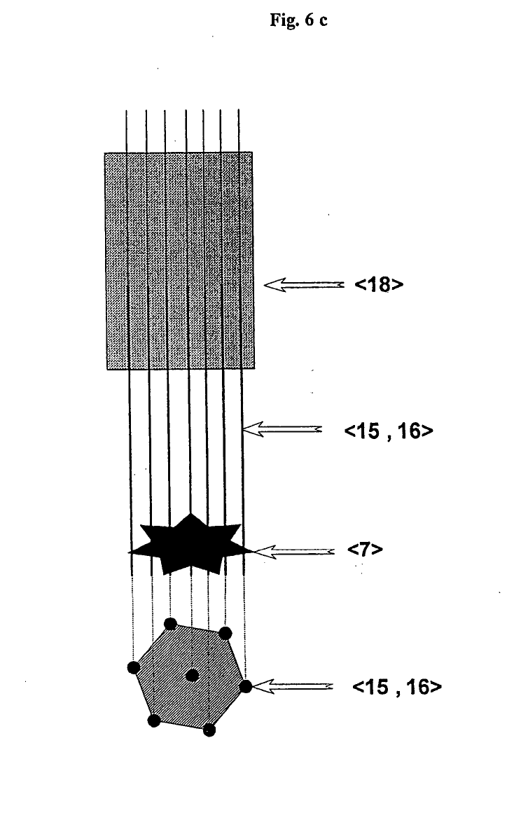

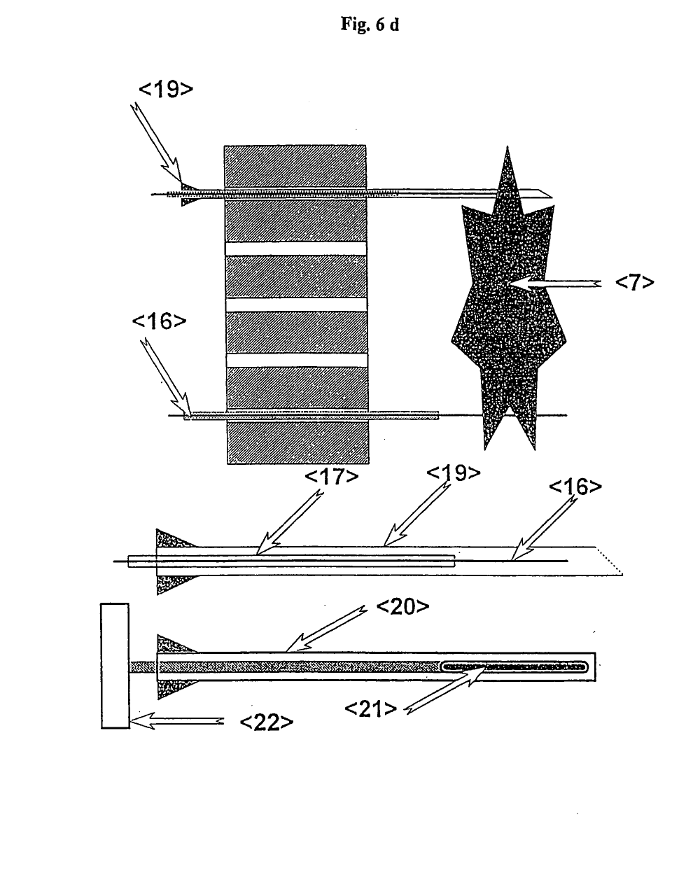

FIGS. 6a-d show embodiments of electrodes and electrode applicators designed for interstitial treatment of tissue;

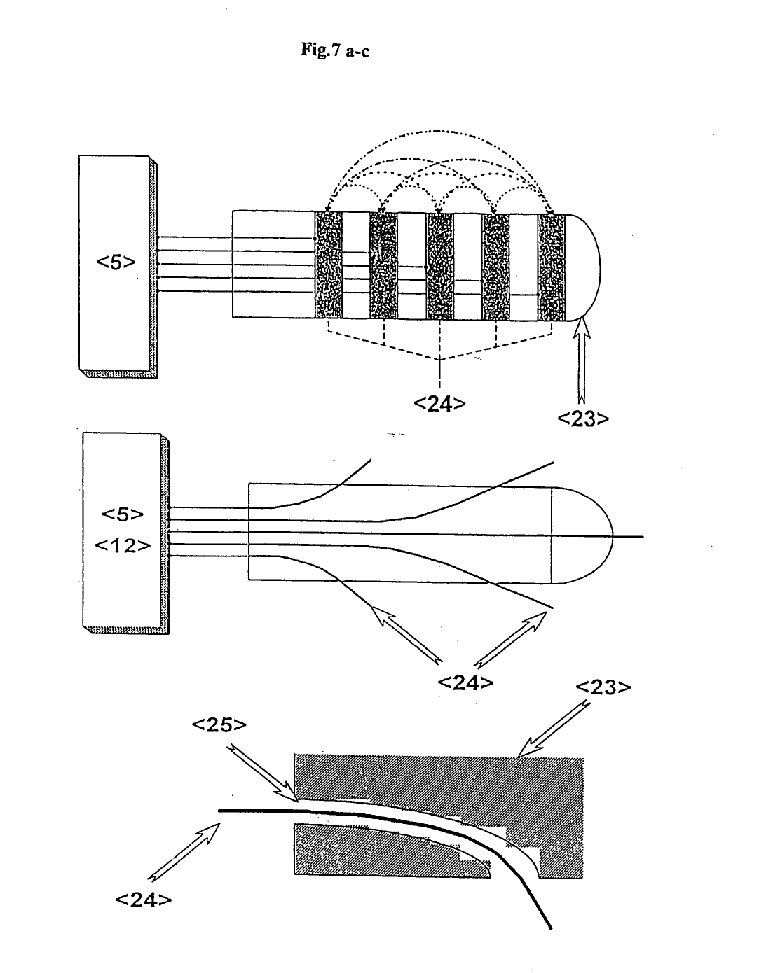

FIGS. 7a-c show embodiments of electrodes and electrode applicators designed for the treatment of, for example, tumors in bodily cavities and in organs accessible via large vessels;

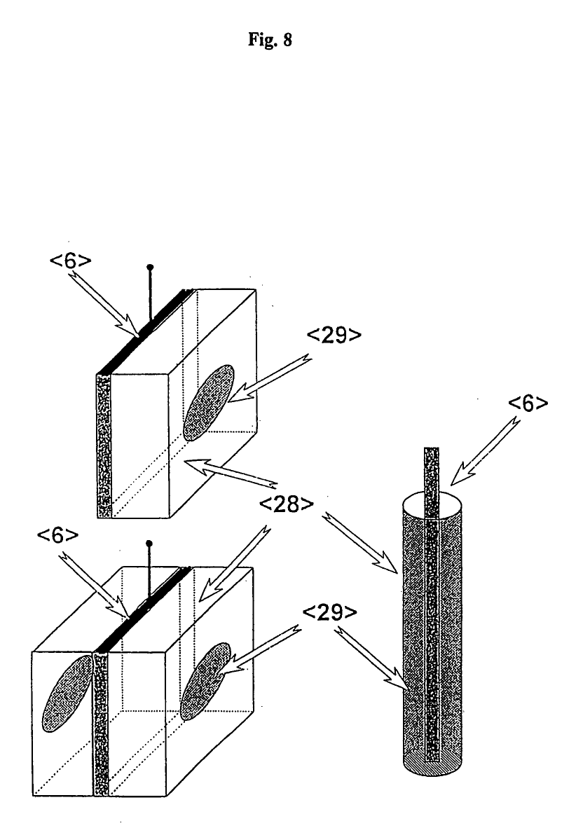

FIG. 8 shows embodiments of electrodes in which these are disposed for combination treatment with antitumoral pharmaceutics;

FIGS. 9a-e show examples of configurations of voltage pulses applied to the electrodes;

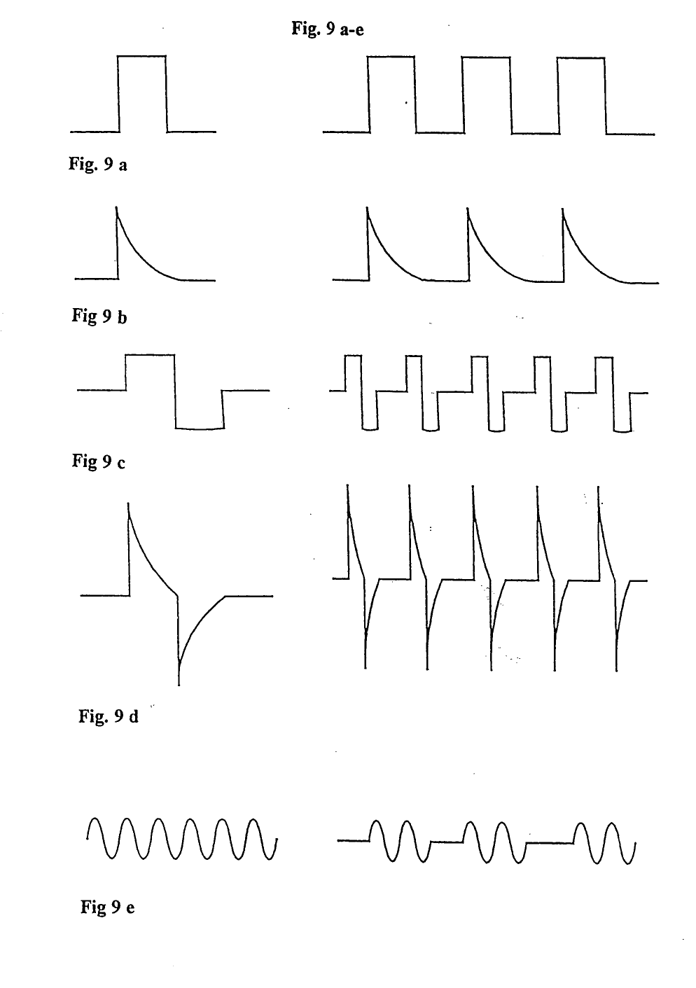

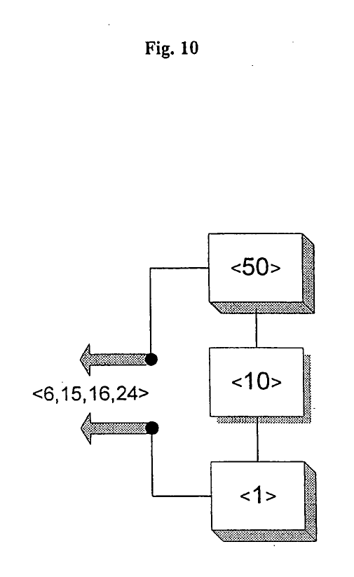

FIG. 10 is a simplified block diagram of one embodiment of the apparatus;

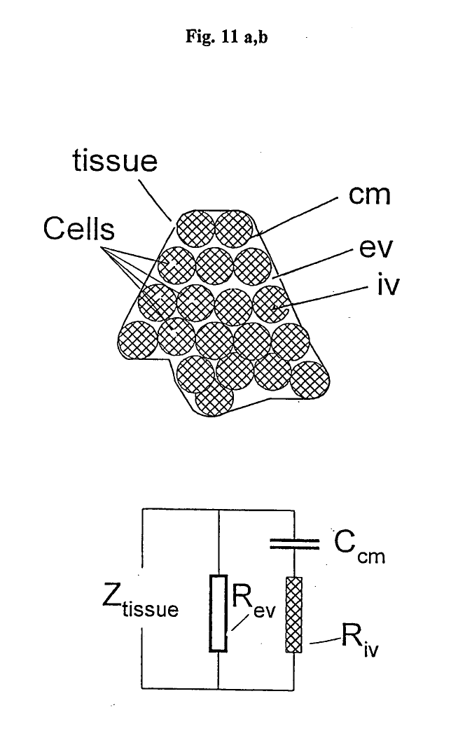

FIG. 11a shows a model of the principle structure of living tissue;

FIG. 11b is an electric skeleton diagram of the electric structure of living tissue; and

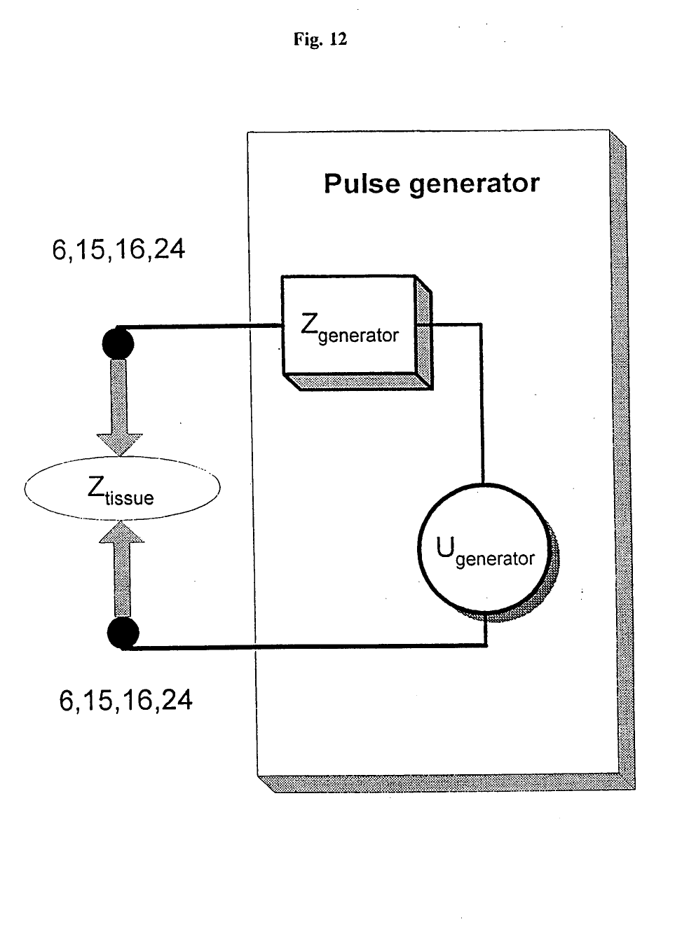

FIG. 12 is an electric model of a pulse generator connected to living tissue.

FIG. 1 shows, in block diagram, the basic design of a high voltage generator 1, electrodes 6,15,16,24 and a registration and conversion device 10, for example a computer or a microprocessor 10, these devices all being included in the apparatus according to the present invention. Hereafter, the word computer will also be employed without any restrictive intent for the registration and conversion device. Between the high voltage generator 1 and the electrodes 6,15,16,24, there are disposed one or more signal connections 32 and electric conductors 33. Between the computer 10 and the high voltage generator 1, and between the computer and the electrodes 6,15,16,24, there is provided one or more signal connections 32. While the signal connections 32 in the Figure are shown as directly connecting the computer and the electrodes, it will be obvious that the apparatus as such also includes devices described in the continued description of this application, such as switches 3, distributor 4, electrode applicator 5, etc. for controlling the voltage impressment of the electrodes, etc.

FIG. 2 shows one embodiment of the present invention in which a radiation transmitter 34 is connected via signal connections 32 to the computer. In certain embodiments, the radiation transmitter is mechanically interconnected to the high voltage generator, while in other embodiments it only has signal connection with the combination of devices illustrated in FIG. 1.

FIG. 3 schematically shows one embodiment of a combination of devices for generating electric fields according to the present invention. The Figure shows blocks for a high voltage generator 1, a capacitor battery 2, a switch 3, a distributor 4 for distributing the high voltage pulses which are generated on discharge of the capacitor battery 2 through the switch 3 to an electrode applicator 5 and electrodes 6 intended to be placed in or adjacent the tissue region 7 or organ 7 of a patient undergoing treatment. The high voltage generator 1, the capacitor battery 2, the switch 3 and the distributor 4 are connected in series with one another by means of electric conductors 33. Between the distributor 4 and the electrode applicator 5, there is provided at least one electric conductor 33 and at least one signal connection 32. Via the signal connections 32, the distributor 4 controls the voltage impression of the electrodes of the electrode applicator, via which the electric conductors 33 are interconnected to the distributor 4 and via the electric conductor 33 to the switch 3. In one alternative embodiment, each electrode 6 is electrically connected to the switch 3 by means of an electric conductor 33.

As a rule, the distributor 4 or an electrode applicator impresses voltage simultaneously on only two electrodes 6, while the other electrodes are permitted to assume that potential which is determined by the placing of the electrode in the treatment region. The term voltage impression also includes in this context the fact that one or more electrodes are earthed (have zero potential). The switch 4 and/or the electrode applicator 5 are disposed to permit, if so wished, the voltage impression pairwise of all electrodes which are placed in the treatment region. It will be obvious to a person skilled in the art, that, in certain embodiments, the devices are provided in order, on voltage impression, to allocate to several electrodes a substantially corresponding (the same) potential.

All units are, via signal connections 32 which, in certain embodiments, are wholly or partly wireless, connected to a registration and conversion device 10 with a screen 10a. Hereafter, the designations control and conversion unit 10 or computer 10 will be employed for the registration and conversion device. The computer 10 constitutes a control and monitoring device for the function of the apparatus.

The expression electrode applicator 5 relates to a retainer member for the electrodes 6, where the retainer member is designed to facilitate the correct application of the electrodes at or in the treatment region.

The computer is set as a rule for the high voltage pulses to contain the following data:

| | |
|---|---|
| repetition frequency | approx. 0.1-10000 per second |
| amplitude | approx. 50-6000 V |
| pulse length | approx. 0.1-200 ms |
| number of pulses | 1-2000 per treatment. |

The pulses are applied before, during or immediately after the radiation treatment. Examples of pulse configuration employed are square pulse with a pulse length of 0.1-2 ms or exponentially fading pulse with a time constant RC approximately equal to 0.1-2 ms. In large amplitudes of the voltage, shorter pulse lengths are generally selected, and vice versa.

The high voltage generator 1 is, as a rule, disposed to emit modulated a.c. voltage of a frequency within a range of 40 Hz-2 MHz and as a rule within the range of 40 Hz-100 kHz. In those embodiments where the high voltage generator is disposed to emit a.c. voltage of high frequency, a modulator is employed instead of a capacitor battery and switch for generating short modulated high frequency pulses with a pulse length within the range of approx. 0.1-200 ms.

As will be apparent from the embodiment illustrated in FIG. 3, the apparatus generally also includes sensors 8 intended to be applied to the patient in the treatment region. The sensors are connected via a detector interface 9 to the registration and conversion device 10. On application of the treatment pulse, a signal is generated in the sensors 8 which, via the interface 9, is transferred to and registered in the computer 10. From the measured signals, the computer calculates the electric field force induced by the pulse and the electromotive force in different parts of the treatment region 7. These signals entail that the computer 10 emits signals to the high voltage generator/capacitor battery (feedback) to adjust the amplitude of the generated pulses so that the predetermined field force is achieved in the treatment region. This monitoring and adjustment take place continuously during the application of the pulses.

FIGS. 4a-d show embodiments of electrode applicators 5 for external treatment of a patient with the electrodes 6 applied in a restricted region on the patient and in different configurations around the tissue region 7, for example a tumor 7, which is to be treated. FIGS. 4a and 4b show how by crosswise application of the electric high voltage pulses to different combinations of two electrodes 6, the result will be achieved, as marked in the Figure by the electric field force lines, that the electric field passes through all parts of the tissue region 7.

FIGS. 4c-d show how electrodes are designed with abutment surfaces of different sizes in order for the field lines to be focused to the desired treatment region. At the beginning of the treatment, the electric high voltage pulses have, for example, a voltage which is adjusted in accordance with the distance between the electrodes. The voltage is then adjusted in accordance with the relationship:
Voltage=(constant)×(the distance between the pairwise electrodes). The value of the constant is varied in response to the type of tissue and is, as a rule, selected at values between approx. 500-3000 V/cm.

Once the treatment has commenced, the control unit and impedance measurement unit described below regulate the output voltage of the pulse regulator to values which entail that the sought-for electric field force passes through the tissue.

FIG. 5 shows one embodiment of an electrode applicator 5 for intraoperative treatment, and treatment of, for example, superficial tumor nodules 7. The electrode applicator has a scissors-like design and comprises two shanks 12 of electrically insulating material (e.g. teflon) which are movably interconnected to one another in a journal 11. The shanks are provided with a gripping lock 13. At one end of each shank 12, the shanks are provided with finger grips and at the other ends with electrodes 6 which grasp about the tumor nodules 7. The grip locks 13 fix the shanks 12 in the set position. The voltage of the electric high voltage pulses is adjusted in response to the size of the tumor 7 with the aid of a distance sensor 14 built into the electrode applicator and connected to the computer 10. The voltage is set at the beginning of the treatment, for example according to the relationship:
Voltage=(constant)×(the distance between the pairwise electrodes). The value of the constant is adapted to the type of tumor and is, as a rule, selected within the range of approx. 500-3000 V/cm.

Once the treatment has commenced, the control unit and the impedance measurement unit described below regulate the output voltage of the pulse generator to values which entail that the sought-for electric field force passes through the tissue.

FIGS. 6a-d show embodiments of electrodes 15,16 and a fixture 18 for the electrodes, where the electrodes and the fixture are suitable for use for interstitial treatment of both superficial and profound tissue. FIG. 6a shows the electrodes 15,16 in two different embodiments, namely in one embodiment in which the electrodes 15 are needle-shaped and in one embodiment in which the electrodes 16 are stiletto-shaped. Each one of the electrodes 15,16 is, in a portion 31 most proximal their one end, provided with an electric conductor 33 for connection to the high voltage generator 1. The above-mentioned portion is provided with an electrically insulating layer 17 or an electrically insulating sleeve 17 in which the electrode is inserted.

The electrodes are applied in different configurations in and about the tissue 7 or the organ 7 which is to be treated, either direct by free hand or with the aid of an electrode applicator (fixture) 18 provided with a hole. The electrode applicator is, as a rule, designed so as to be removed from the electrodes 15,16 once these have been applied on the patient. It will thereby be possible to allow the electrodes to remain in position in the patient to be used on several subsequent treatment occasions. Alternatively, the electrode applicator is removed together with the electrodes 15,16 after each treatment. Also in interstitial treatment, there are electrodes with surfaces of different sizes for controlling the extent of the electric fields.

Those parts of the electrodes 15,16 which are intended to be inserted into the patient to cover the extent of the tissue 7 which is to be treated are, for example, manufactured of stainless steel of a quality which agrees with or corresponds to that employed for injection syringes or are manufactured or coated with another tissue-friendly metal such as a noble metal, for example gold or platinum. The remaining portion of the electrodes forms an insulated portion 17 with input conductors 33 for the high voltage pulses. On the employment of flexible input conductors, the electrode is placed in a large cannula 19 which, after application of the electrode in the patient, is withdrawn, the electrodes remaining in position in the tissue.

In certain embodiments, the electrodes consist of radioactive metal (e.g. iridium-192, cobalt-60) or are surface coated with radioactive substances (e.g. iodine-125). In other embodiments, they are designed as tubes 20 of inert metal which are charged with radioactive material (e.g. ¹⁹²Ir, ¹³⁷Cs, ²²⁶Ra) which advantageously takes place by the employment of a so-called after loading device 22. The pulses have a voltage which, at the start of the treatment, for example are determined by the distance between the electrodes. The voltage is then set according to the relationship:
Voltage=(constant)×(the distance between pairwise electrodes). The value of the constant is selected in response to the type of tumor and, as a rule, selected within the range of approx. 500-3000 V/cm.

Once the treatment has commenced, the control unit and the impedance measurement unit described below regulate the output voltage of the pulse generator to values which entail that the sought-for electric field force passes through the tissue.

In those applications where treatment with electric fields is combined with radiation treatment from a radiation source which is located outside the treatment region, the electrodes in the treatment region are supplied with electric voltage pulses before, during or immediately after the radiation treatment.

FIGS. 7a-c show electrodes 24 for treating tissue accessible via, for example, major vessels, or via bodily cavities, for example respiratory tracts, urinary tracts and stomach-intestinal trait. The electrodes are disposed on the surface of a cylinder-like electrode applicator 23 of insulating material 17. In certain embodiments, the electrodes are designed such that they are introduced into the tissue through channels 25 in the applicator 23 operated by a remote control. As will be apparent from FIG. 7c, the channels 25 (according to the embodiment described in the preceding sentence) discharge in the circumferential surface of the electrode applicator, whereby the electrodes 24 are, on their displacement, guided into tissue surrounding the electrode applicator. In certain embodiments, the applicator is disposed to be supplied with radioactive preparations, whereby the applicator also forms a radiation device. The applicator is disposed to be supplied with the radioactive preparations manually or by means of an after loading device 22. The voltage of the electric high voltage pulses is adjusted during the treatment.

The field lines in FIG. 7a indicate the extent of the electric field lines in the tissue.

For intracavity treatment of tissue in different, irregularly shaped bodily cavities (e.g. oral cavity, respiratory tracts, oesophagus, stomach, uterus, bladder, ureter, rectum) electrode applicators 23 are applied as is apparent from FIGS. 7a-c, particularly designed in response to the configuration of the cavity, with electrodes applied on the surface 24 or alternatively designed as needles which, through channels 25, are passed into the tissue by remote control. These applicators are suitable for use when treating, for example, lung cancer, liver tumors, renal tumors and tumors in the stomach-intestines with reduced absorbed dose for reducing side effects of the radiation treatment in normal tissue. Prostate cancer is treated with applicators applied via the rectum and the ureter. These applicators are, in certain embodiments, designed to be charged with radioactive sources or radioactive material 21, either manually or using an after loading device 22.

FIG. 8 shows an apparatus for combined treatment with antitumoral pharmaceutics where the electrode 6 is coated with a layer 28 of porous metal, glass, ceramics, inert plastic or other polymer which contains antitumoral pharmaceuticals 29 (e.g. bleomycin, platinol, taxol, monoclonal antibodies), genetic material (chromosomes, DNA) or radioactive substances (e.g. iodine 125, Auger-electron emitters) 29. This type of electrode is well suited for use in radiation therapy, since the high electric field force increases the permeability of the tumor cell for the above mentioned substances and thereby increases the antitumoral effect.

FIGS. 9a-e show examples of pulse forms in the voltage pulses which are pairwise applied to the electrodes 6,15,16,24. In the Figures the height of the pulse represents the voltage between two electrodes. The width of the pulse represents the length of the pulse. The FIGS. 9a and 9c show examples of square pulses, FIGS. 9b and 9d examples of pulses whose voltage fades with time, and FIG. 9e pulses of alternating voltage. FIGS. 9c and 9d show voltage pulses where, analogous to that which applies in alternating voltage, the electrodes alternatingly have the highest voltage, whereby a corresponding change takes place of the electric field between the electrodes.

The above described electrodes 6,15,16,24, the voltage generator 1, the control and converter device 10, also previously designated the computer and an impedance measurement unit 50 are included in the block diagram shown in FIG. 10. The voltage generator, the computer, the electrodes and the impedance measurement unit are interconnected with one another by electric conductors for impressing voltage on the electrodes and for transferring signals. It will be obvious to a person skilled in the art that, in certain embodiments, at least a part of the signal connections are designed as wireless connections.

FIG. 11a shows the basic structure of living tissue, while FIG. 11b shows an electric outline diagram for the electric structure of the tissue. The correspondences between the resistances and the capacitance in the electric diagram and in the tissue are apparent from the designations of the components and the continued description.

FIG. 12 shows the basic electric structure of a pulse generator 1, previously also designated high voltage generator. The Figure shows how the impedance of the tissue Zₜᵢₛₛᵤₑ via the electrodes 6,15,16,24 is connected in series to the inner impedance of the pulse generator Zgenerator. Reference letter U relates to electromotive force (EMF) of the pulse generator.

It will be obvious to a person skilled in the art that the above described mechanical units, in certain embodiments of the present invention, form mutually separate mechanical units which are inter-connected with each other by means of electric conductors and signal connections, while, in other embodiments, some or all of these units, with the exception of the electrode applicator and the electrodes, form a mechanical unit which is co-ordinated with the voltage generator, the impedance measurement unit or the computer.

As will have been apparent from the foregoing description, the present invention relates to an apparatus for high voltage impulse therapy (HVIT) with detection of the treatment effect. The apparatus includes an impedance measurement unit which, on treatment of tissue or organs, is employed for measuring the electrical impedance of the tissue. The impedance measurement unit is, as a rule, disposed to measure the impedance of the tissue at, at least, one frequency. Normally, the impedance measurement unit is disposed to measure the impedance of the tissue within a frequency range, e.g. within the range of 10 Hz to 10 Mhz. With the aid of a mathematical algorithm, a test magnitude is calculated whose value is a measurement of the treatment effect.

The voltage across the tissue will be in accordance with that shown in FIG. 12:
Uₜᵢₛₛᵤₑ=U_{generator}*Zₜᵢₛₛᵤₑ/(Zₜᵢₛₛᵤₑ+Z_{generator})

The impedance of the tissue varies extremely, depending upon the cell structure and build up of the tissue, the nature of the surrounding tissue and the quantity of bodily liquids which are found in and around the treated region. Since the output impedance of the generator is not slight in relation to the impedance of the tissue, the output voltage will vary greatly depending upon where and how the applicator is placed. It has proved, in practical experiments, that even if an applicator is placed at the same point, marked with a colour on the body, the impedance will vary greatly from time to time, depending upon minor differences in placing and contact impedance, as well as differences in fluid quantity and the nature of the tissue.

In order to be able to predict the actual pulse voltage from the pulse generator, the impedance of the tissue must be known at any time. Only if the output voltage from the generator is adjusted on the basis of the generator's output impedance and the impedance of the relevant tissue will it be possible to achieve a predictable and constant effect. According to the present invention, the apparatus includes means for measuring the impedance of the treated tissue and means for employing this information for controlling the output voltage of the pulse generator such that the desired field force is always achieved in the tissue.

FIG. 10 illustrates such a system. A control unit is included in the apparatus and measures, with the aid of the impedance measurement unit, the impedance of the tissue. The control unit adjusts the output voltage from the generator so that the desired field force is achieved.

In the control unit, which, for example, is a PC, the desired field force is set whereafter the control unit measures the impedance in the tissue and calculates the requisite pulse voltage from the generator. When a pulse is subsequently applied, the field force will always be constant, since the control unit always measures and adjusts the voltage from the generator before the pulse is generated.

With the system in FIG. 10 the sought-for effect will be achieved, e.g. to maintain a constant output voltage from the pulse generator independently of the impedance in the tissue. It also proves that a system according to FIG. 10 is excellent for measuring and assessing the treatment result achieved in HVIT. By measuring impedance and carrying out analysis of impedance change in the tissue after a pulse has been applied, the documentary support is given for assessing when the treatment is completed and no more pulses are needed or give a further positive effect. This method builds on the tissue model shown in FIG. 11a,b.

The impedance in tissue substantially consists of three components, the resistance in the extra cellular fluid, the resistance in the intra cellular fluid and the capacitance which is formed between the D.C. insulating effect of the cell membrane. In the model, we have combined the impedance effect of the cell core with the resistance in the intra-cellular fluid. At low frequencies, only current will flow through the extra cellular liquid and the impedance is determined substantially by Rₑᵥ. At medium-high frequencies, the capacitance of the cell membrane C_{cm} together with the resistance of the intracellular liquid, Rᵢᵥ, will begin to effect the impedance. At high frequencies, substantially the components Rₑᵥ and Rᵢᵥ will effect the impedance of the tissue. Thus, the result will be a frequency dependence in the impedance of the tissue which is largely dependent on the thickness of the cell membrane and the formation of the cells. At low frequencies, the impedance is approximately Rₑᵥ and at high frequencies Rₑᵥ//Rᵢᵥ. The symbol // is employed to indicate that Rₑᵥ is connected in parallel with Rᵢᵥ.
Zₜᵢₛₛᵤₑ=Rₑᵥ//(Rᵢᵥ+C_{cm})

Since the treatment with electric fields is intended to render the cell membrane permeable or to wholly destroy it, a clear indication will be obtained by measuring the change in C_{cm} as to whether the treatment is completed or not. When all cell membranes in the tissue are destroyed, no change of C_{cm} will take place any longer and the tissue is ready-treated.

Table 1 below illustrates a compilation of impedance measurements taken during the treatment of rats with tumors.

TABLE 1
Measured tissue impedance in ohm in rats with tumor

| | | 0 | 16 | 32 | 48 | 64 |
|---|---|---|---|---|---|---|
| Frequency/Pulses | | pulses | pulses | pulses | pulses | pulses |
| 10 | Hz | 232.24 | 160.12 | 160.36 | 172.53 | 179.3 |
| 15 | Hz | 229.42 | 157.76 | 151.48 | 163.37 | 159.61 |
| 20 | Hz | 200.28 | 145.46 | 138.84 | 148.89 | 141.78 |
| 30 | Hz | 173.9 | 134.11 | 127.56 | 132.16 | 125.87 |
| 50 | Hz | 153.7 | 122.75 | 116.44 | 120 | 112.29 |
| 70 | Hz | 144.46 | 116.39 | 110.38 | 136.26 | 105.58 |
| 100 | Hz | 137.64 | 110.69 | 105.13 | 105.47 | 100.31 |
| 150 | Hz | 130.68 | 104.86 | 99.79 | 99.71 | 95.35 |
| 200 | Hz | 125.81 | 100.97 | 96.31 | 96.23 | 92.26 |
| 300 | Hz | 120.3 | 96.27 | 92.06 | 92.19 | 88.73 |
| 500 | Hz | 113.96 | 91.09 | 87.49 | 87.84 | 84.91 |
| 700 | Hz | 109.83 | 87.88 | 84.68 | 85.16 | 82.6 |
| 1000 | Hz | 105.88 | 85.03 | 82.2 | 83.03 | 80.62 |
| 1500 | Hz | 101.99 | 82.12 | 79.71 | 81.84 | 78.59 |
| 2000 | Hz | 99.34 | 80.27 | 78.02 | 79.54 | 77.69 |
| 3000 | Hz | 96.12 | 77.98 | 76.06 | 77.18 | 75.72 |
| 5000 | Hz | 92.28 | 75.4 | 73.81 | 74.85 | 73.71 |
| 7000 | Hz | 89.72 | 73.85 | 72.41 | 73.86 | 72.54 |
| 10000 | Hz | 87.38 | 72.45 | 71.14 | 73.52 | 71.43 |
| 15000 | Hz | 84.91 | 70.91 | 69.71 | 72.53 | 70.15 |
| 20000 | Hz | 83.18 | 69.75 | 68.62 | 71.51 | 69.17 |
| 30000 | Hz | 80.8 | 68.23 | 67.14 | 69.81 | 67.8 |
| 50000 | Hz | 77.73 | 66.26 | 65.28 | 68.24 | 65.97 |
| 70000 | Hz | 75.62 | 64.79 | 63.9 | 66.67 | 64.65 |
| 100000 | Hz | 73.01 | 63.01 | 62.11 | 64.62 | 62.93 |
| 150000 | Hz | 70.42 | 61.05 | 60.3 | 64.06 | 61.19 |
| 200000 | Hz | 68.3 | 59.37 | 58.76 | 61.93 | 59.65 |

It will be apparent from Table 1 that the impedance reduces at low and medium-high frequencies after treatment with pulses. The reduction principally takes place after the introductory 16 pulses and the change rapidly fades thereafter. Thus, the rat is substantially ready-treated already after the first 16 pulses and further treatment after 32 or 48 pulses gives no major change in C_{cm}. The measurement data in Table 1 indicates that the treatment is completed after 32 pulses. In order to confirm this assessment, the measured measurement values have been taken and treated as described below.

Table 2 shows the impedance change in percent at different frequencies after the electric fields generated by 16 voltage pulses have passed through the tissue. In the Table, the change of the impedance is given in percent which occurred each time when a series of electric fields generated by the voltage pulses has passed through the tissue.

TABLE 2
Impedance change in percent after
treatment with 16 pulses at a time

| Frequency/Pulses | | 16 pulses | 32 pulses | 48 pulses | 64 pulses |
|---|---|---|---|---|---|
| 10 | Hz | −31.05408 | 0.1033414 | 5.2402687 | 2.9150878 |
| 15 | Hz | −31.23529 | −2.737338 | 5.1826345 | −1.638916 |
| 20 | Hz | −27.37168 | −3.305372 | 5.0179748 | −3.55003 |
| 30 | Hz | −22.88097 | −3.766532 | 2.6451984 | −3.617021 |
| 50 | Hz | −20.13663 | −4.1054 | 2.3162004 | −5.016265 |
| 70 | Hz | −19.43098 | −4.160321 | 17.914994 | −21.23771 |
| 100 | Hz | −19.58006 | −4.039523 | 0.2470212 | −3.74891 |
| 150 | Hz | −19.75819 | −3.879706 | −0.061218 | −3.336394 |
| 200 | Hz | −19.74406 | −3.703998 | −0.063588 | −3.155552 |
| 300 | Hz | −19.97506 | −3.499584 | 0.1080632 | −2.876143 |
| 500 | Hz | −20.06845 | −3.159003 | 0.3071253 | −2.571078 |
| 700 | Hz | −19.98543 | −2.913594 | 0.4370391 | −2.330875 |
| 1000 | Hz | −19.6921 | −2.672837 | 0.7839063 | −2.276162 |
| 1500 | Hz | −19.4823 | −2.362977 | 2.08844 | −3.186587 |
| 2000 | Hz | −19.1967 | −2.264949 | 1.5300987 | −1.862291 |
| 3000 | Hz | −18.87224 | −1.997503 | 1.1652102 | −1.518935 |
| 5000 | Hz | −18.29215 | −1.723017 | 1.1270048 | −1.235371 |
| 7000 | Hz | −17.68836 | −1.604993 | 1.6161391 | −1.471244 |
| 10000 | Hz | −17.08629 | −1.499199 | 2.7237354 | −2.391852 |
| 15000 | Hz | −16.48805 | −1.413261 | 3.3211636 | −2.802968 |
| 20000 | Hz | −16.14571 | −1.3585 | 3.4743929 | −2.813176 |
| 30000 | Hz | −15.55693 | −1.34901 | 3.3044554 | −2.487624 |
| 50000 | Hz | −14.75621 | −1.260774 | 3.8080535 | −2.920365 |
| 70000 | Hz | −14.32161 | −1.176937 | 3.6630521 | −2.671251 |
| 100000 | Hz | −13.69675 | −1.232708 | 3.4378852 | −2.314751 |
| 150000 | Hz | −13.30588 | −1.065038 | 5.3393922 | −4.075547 |
| 200000 | Hz | −13.07467 | −0.893119 | 4.6412884 | −3.338214 |

The heading of the Table discloses the accumulated number of pulses of electric fields which have passed through the tissue. On each treatment occasion, a series of 16 pulses is passed through the tissue. That disclosed in this paragraph for the table heading in Table 2 also applies to the table headings for Tables 3 and 4 used below.

It will be apparent from Table 2, in the same manner as Table 1, that the treatment may be discontinued after 32 pulses, since the impedance change fades dramatically. Table 3 below shows the mean value of the impedance change after different numbers of pulses. The mean value is formed from all measurement frequencies between 10 Hz and 200 kHz. In Table 3, it is clearly seen that the largest impedance change takes place after the first 16 pulses and only a slight change takes place on further treatment.

TABLE 3
Progressive change in percent of impedance value
at frequencies between 10 Hz-200 kHz

| 16 pulses | 32 pulses | 48 pulses | 64 pulses |
|---|---|---|---|
| −19.9568 | −2.424687 | 3.1275358 | −3.366544 |

In Table 4, in mean value formation, frequencies below 100 Hz and frequencies over 10 kHz have been deleted. By deleting the lowest frequencies from the mean value, this prevents incorrect impedance values because of disturbance from the motorsystem of the body from influencing the result. The highest frequencies are deleted since the impedance change at these high frequencies is less when C_{cm} is changed and therefore does not contribute to an improved picture of the treatment result.

TABLE 4
Progressive change in percent of impedance values
at frequencies between 100 Hz-10 kHz

| 16 pulses | 32 pulses | 48 pulses | 64 pulses |
|---|---|---|---|
| −20.78512 | −2.943407 | 1.0007481 | −2.663449 |

By allowing the control unit in FIG. 10 mathematically to treat and present the measured treatment result as described above, there will be obtained an apparatus which satisfies the wishes of controlling, in the treatment, the strength of the electric field in order to obtain a basis for discontinuing the treatment at the correct moment and for being able to interpret the direct outcome of the treatment with the electric field.

From the foregoing description, it will be apparent that, in a very simple application of the present invention, the impedance of the tissue is determined at only one frequency. In such instance, a medium-high frequency, e.g. 15 kHz is selected. The inner impedance of the pulse generator is entered in the computer as a fixed value, whereby the impedance of the tissue is determined by a mathematical operation corresponding to that described above. In applications of the present invention, use is however made as a rule of many frequencies in order to eliminate the risks of any possible disruptions which may affect the measurement results.

The system illustrated in FIG. 10 includes means for adjusting the pulse voltage and its frequency content so that the electric field in the treated tissue is always constant regardless of impedance or resistance changes in the tissue. Such means also give a basis for assessing the achieved treatment effect in that it is of a structure which makes it possible to present, for example readily understandable values and graphs which, by mathematical operations, have been extracted from measured impedance or resistance data.

On practical application of the present invention in the embodiment where a radiation transmitter is employed, the radiation transmitter and the electrodes, in certain applications together with the electrode applicator and impedance measurement unit, together form a cohesive mechanical unit. This is of a design which makes it possible, in a restricted region of a human or an animal, to apply both the radiation transmitter and the electrodes in positions where the ionizing radiation is directed at the tissue which is being treated and where the electrodes are in positions in which electric fields between them pass through the tissue. In other embodiments, such means constitute separate parts which, together and where applicable temporarily, or for a lengthy period of time, form a system of devices of a composition corresponding to that described above for the apparatus 40.

The above detailed description has referred to but a limited number of embodiments of the present invention, but a person skilled in the art will readily perceive that the present invention encompasses a large number of embodiments without departing from the scope of the appended claims.

1. An apparatus for treating tissue or organs, said apparatus comprising:
a plurality of electrodes arranged in an electrode applicator adapted for placement within a restricted region of the tissue or organ,
a high voltage generator arranged to generate and deliver one or more high voltage pulses to said plurality of electrodes,
an impedance measuring means arranged between the plurality of electrodes for measuring impedance within said restricted region, at at least one of before, during and after application of said one or more high voltage pulses, at at least one frequency, and
a registration and conversion means, arranged between said high voltage generator and said impedance measuring means for receiving signals from the impedance measuring means and emitting signals to the high voltage generator to control the pulses produced thereby based on the impedance measured in said restricted region.

2. The apparatus according to claim 1, wherein said high voltage generator generates the high voltage pulses at a value high enough to produce cell electroporation.

3. The apparatus according to claim 1, wherein said one or more high voltage pulses is set to a repetition frequency of approximately 0.1 to 10000 cycles per second.

4. The apparatus according to claim 1, wherein said electrodes are in the form of needles or stilettos.

5. The apparatus according to claim 1, wherein said impedance measuring means comprises sensors for application to the tissue or organ within said restricted region.

6. The apparatus according to claim 1, wherein the pulses produced by the high voltage generator are controlled by the registration and conversion means to produce a substantially constant pre-determined electric field in said restricted region of the tissue or organ.

7. The apparatus according to claim 1, wherein the impedance measuring means being arranged between the plurality of electrodes for measuring impedance at a plurality of frequencies.

8. A method for implementation in an apparatus for treating tissue or organs having a plurality of electrodes adapted for placement within a restricted region of the tissue or organ, a high voltage generator arranged to generate one or more high voltage pulses to said plurality of electrodes, an impedance measuring unit arranged between the plurality of electrodes to measure impedance before, during and after application of said one or more high voltage pulses at at least one frequency, and a registration and conversion device communicating with said impedance measuring unit and said high voltage generator to receive impedance measurements measured by the impedance measuring unit and to control said high voltage generator, said method comprising the steps of
receiving an initial impedance measurement measured by the impedance measuring unit before the one of more high voltage pulses to be generated by the high voltage generator,
storing the impedance measurements received in the step of receiving an initial impedance measurement,
emitting an initial control signal to the high voltage generator to generate a high voltage pulse based on the impedance measurement received in the step of receiving an initial impedance measurement,
receiving an intermediate impedance measurement measured by the impedance measuring unit during the one or more high voltage pulses to be generated by the high voltage generator,
storing the impedance measurement received in the step of receiving an intermediate impedance measurement,
obtaining an assessment by assessing effects of the one or more high voltage pulses generated by the high voltage generator by comparing the initial impedance measurement stored in the step of storing the initial impedance measurement and the intermediate impedance measurement stored in the step of storing the intermediate impedance measurement, and
emitting a signal to the high voltage generator to adjust or terminate the high voltage pulses base on the assessment obtained in the step of obtaining an assessment.
